(19) United States
(12) Patent Application Publication
(10) Pub. No.: US 2022/0005576 A1
(43) Pub. Date: Jan. 6, 2022

(54) SYSTEM AND METHOD FOR LINKING A PRODUCT TO PRODUCT INFORMATION

(71) Applicant: Vive Health LLC, Naples, FL (US)

(72) Inventors: Matthew Fleming, Naples, FL (US); Aaron Gaunt, Estero, FL (US); Anthony Henry Barbier, Bonita Springs, FL (US)

(21) Appl. No.: 17/368,263

(22) Filed: Jul. 6, 2021

Related U.S. Application Data
(60) Provisional application No. 63/047,955, filed on Jul. 3, 2020.

(51) Int. Cl. G16H 20/30; G06F 3/0484; G06K 7/14; G06N 20/00; A63B 24/00

(52) U.S. Cl. CPC G16H 20/30; G06F 3/0484; G06F 3/0482; G06N 20/00; A63B 24/0075; G06K 7/1417

(57) ABSTRACT

A system and method of linking a product to product information comprises a physical product configured for use by a user and a scannable identifier associated with the physical product. The scannable identifier is accessible to the user, and the scannable identifier is configured to cause a user device to navigate to content related to use of the physical product in response to scanning of the scannable identifier with a scanning sensor associated with the user device.

### CROSS-REFERENCE TO RELATED APPLICATIONS

This application claims priority to and the benefit of U.S. Provisional Patent Application Ser. No. 63/047,955, filed Jul. 3, 2020, and entitled “System and Method for Linking a Product to Product Information,” which is incorporated herein by reference in its entirety.

### BACKGROUND OF THE INVENTION

### 1. The Field of the Invention

Embodiments of the present disclosure relate generally to systems and methods for linking products to product information.

### 2. Background

Many products and services require a user to have access to information to properly and safely use the product or service. For example, a user cannot properly and safely use a medical device without accurate information regarding how the medical device functions in relation to the user's indications. In another example, a user may not be able to intuit the operating details of an electronic device or locomotive device.

Because many users purchase products online and do not receive training from a salesperson or technical representative, such information may not be readily accessible to a user. Even when products or services are sold in-person, the transaction may take place at a third-party vendor where trained persons are not available to provide instruction on how to properly use the product, or training may be shallow or subpar. Even when adequate training is provided, the user may require updated or different information later as the user uses the product in different ways or has questions.

The COVID-19 pandemic, which has shuttered in-person business and transactions around the world, has exacerbated the problem of users of a product or service not having access to training or product information, as many goods are being ordered online and delivered without any person-to-person contact. Products or services directed to physical fitness and/or physical rehabilitation are examples of types of products and services that can be purchased through the internet but may require some expertise to know exactly the best methods of use. Additionally, many websites and product catalogues are uneasy to navigate, such that a user may not be able to access necessary information, let alone properly interpret and utilize the information, on their own.

While products or services may be delivered to a user with an instruction manual detailing certain steps for use and/or precautions, there is a problem of existing modalities for delivering or otherwise providing products to users that do not provide a user with a reliable and/or customized source of information regarding the product.

Furthermore, many instances exist where a user uses a product that the user did not purchase. For example, when a user uses a product owned by another person or entity, the user may lack access to instructional and/or other information related to proper use of the product.

There is a need for an improved method and system for linking products or services to product information.

### BRIEF SUMMARY

The present disclosure is directed to embodiments of a system and/or method for linking a product to product information. The disclosed embodiments address the problem of existing modalities for providing a product to a user that lack a way for the user to access important information regarding the product. Embodiments of the disclosed system and/or method enable a user to access a customizable library of information and interactive features regarding a product in a simplified manner.

The system and method may provide, in at least one embodiment, an identifier on at least one product or service from a vendor. The identifier may be a scannable element, such as a Quick Response (QR) code, a barcode, or other scannable element that a user may scan using a suitable modality on a mobile phone, for example. The identifier may be applied to the product on an exterior surface thereof, such as on a box or packaging of the product. The identifier may be linked to information pertaining to the specific product that the user purchased (or that was purchased by another and that is currently being used by the user).

For example, the user may have purchased a medical device such as a crutch (or may otherwise be using a crutch purchased by another person or entity). The user may scan the identifier when the crutch is provided to the user, which may navigate the user to a location on the vendor's website or mobile application corresponding to the medical device. The website or mobile application may contain a library comprising information regarding the crutch, for example videos demonstrating how the crutch is to be used, workouts with instructions of how to exercise with or in order to better use the crutch, or otherwise. This can advantageously help the user avoid applying incorrect information to the product provided to the user (e.g., information from the vendor's website or mobile application related to other products that the user might misapply to the product provided to the user).

In embodiments, the identifier, upon being scanned, may facilitate navigation (e.g., of a scanning device of the user) to a mobile application store, such as the Apple App Store or Google Play, if the vendor's mobile application is not already installed on the user's device, to prompt the user to install the application. If the application is already installed, the identifier upon being scanned may automatically navigate the user to the pertinent location within the application.

The website or application may be personalized to the user based at least in part on one or more identifiers that have been scanned by the user. For instance, the user may purchase more than one product or service from a vendor, each of which may comprise a unique scannable identifier. The user may scan each of the identifiers, and the mobile application or website may be customized to the user based on the identifiers scanned (or based on the products purchased), with the information or content pertaining to the products associated with the scanned identifiers (e.g., the purchased products) prioritized over other content.

In embodiments, the product is a medical device for rehabilitation, such as a strap for stretching one or more body parts, and the information or content provided to a user upon scanning the identifier comprises a suggested workout and/or a library of possible workouts that may be performed using the strap.

The suggested workout and/or the library of possible workouts may include one or more videos of physical therapists demonstrating the workout and/or giving instructions. For example, the video may include the physical therapist explaining when the workout is indicated for a user. In embodiments, the physical therapist may explain that if the user's calf hurts in a particular way, the strap may be wrapped about the user's foot and pulled in a particular direction, magnitude, and duration so as to stretch the calf to alleviate the pain.

Other videos and/or workouts may include, for example, coach workouts where a coach such as a personal trainer demonstrates and/or leads a workout. This may include aerobic exercise including group fitness classes such as dance aerobics classes like Zumba, boot camp classes, spinning classes, and etc., or solo aerobic exercise done on a piece of exercise equipment, such as a rowing machine, recumbent bike, pedal exerciser, x-bike, treadmill, or otherwise. The workouts may alternatively or in addition comprise strength training exercise, including weightlifting, calisthenics, and strength training done on a piece of exercise equipment; balance or flexibility training, such as yoga, pilates, or otherwise. Other types of workouts are envisioned, and the disclosure is not limited to the depicted embodiments.

The information or content provided by the application may include one or more of fitness information and/or rehabilitation information, allowing a user to utilize the product for general fitness purposes, and/or for rehabilitation of a particular issue.

The information or content provided by the website or application may change over time in response to the user's engagement therewith. The website or application may utilize an artificial intelligence modality such as a machine learning model to adapt the content provided to the user upon scanning an identifier or upon using the website or application without scanning an identifier.

The machine learning model may utilize engagement information from a plurality of users to customize information or content provided to a particular user. For example, it may be determined using the machine learning model that users of a particular class of products respond more positively to videos demonstrating workouts than to step-by-step guides, leading to videos being prioritized over non-video guides.

The website or mobile application may be configured to inquire a user's location, such as by utilizing the location features of the user's mobile device or by prompting the user to enter their location manually. The location information may be used to further customize the information or content presented to the user. For example, it may be determined by the machine learning model that users in that particular location, such as a state of the United States, or a particular county, respond better to content presented in a particular way.

In another embodiment, the website or mobile application may provide a screen showing a picture of a body allowing the user to click on or otherwise identify a part of the body that hurts. The user's selection may be used to further customize the content or information provided to the user. Other types of user input may be used to facilitate different customizations to the content or information provided to the user (e.g., a user may input a particular injury or condition that the user desires to utilize a product to treat, and the input may cause modifications to presented content related to the product).

The website or mobile application may further be configured to help the user to utilize the one or more products in a consistent and/or recommended manner. In embodiments, the product may be a medical device configured to allow the user to perform certain exercises, such as for general fitness and strengthening of muscle groups, or for rehabilitation of a joint or muscle group. The application may help the user to track their usage of the device, including providing fields for the user to enter information regarding individual workouts (or by automatically populating certain fields with information obtained by a user device, such as information based on sensor data obtained by one or more sensors of the user device, such as one or more accelerometers, gyroscopes, magnetometers, cameras, microphones, and/or others). The fields may include information relating to duration of the workout, calories burned, distance covered, etc.

In embodiments, if a user views a workout-related video from the content provided by the application, the application assumes that the user completed the workout and may automatically log a corresponding amount of workout duration, calories, and distance covered.

On a home page or screen of the website or application, statistics regarding a user's progress relating to certain metrics may be shown. The metrics may include calories burned, distance covered, and time spent performing activities such as exercises. The metrics may be measured on a daily, weekly, or other basis and may be measured against a standard or custom goal. The user's progress along one or more of the metrics may be shown in a color-coded fashion. For example, the metrics may progress from a color indicating poor progress (such as red) along a pre-defined spectrum toward a color indicating good progress (such as green). The metrics shown on the home page or screen may be standard and automatic or may be customized for or by the user, such as based on the product and corresponding identifier scanned by the user.

In embodiments, the user may purchase a shoulder pulley device. Upon scanning the identifier provided with the product upon delivery, the mobile application for the user may automatically adjust to show a custom arrangement of metrics, including time spent, repetitions completed of specified exercises, or otherwise.

### BRIEF DESCRIPTION OF THE DRAWINGS

These and other features, aspects, and advantages of the present disclosure will become better understood regarding the following description, appended claims, and accompanying drawings.

FIG. 1 illustrates example components of a system for linking a product to product information.

FIG. 2A depicts an example embodiment of a home page of a mobile application of a system for linking a product to product information according to the present disclosure.

FIG. 2B depicts another example embodiment of a home page of a mobile application of a system for linking a product to product information according to the present disclosure.

FIG. 3A depicts an example embodiment of a content interface of a mobile application of a system for linking a product to product information according to the present disclosure.

FIG. 3B depicts an example embodiment of a profile interface of a mobile application of a system for linking a product to product information according to the present disclosure.

FIG. 4A depicts an example embodiment of a workout type selection interface of a mobile application of a system for linking a product to product information according to the present disclosure.

FIG. 4B depicts an example embodiment of a workout selection interface of a mobile application of a system for linking a product to product information according to the present disclosure.

FIG. 4C depicts an example embodiment of a workout context selection interface of a mobile application of a system for linking a product to product information according to the present disclosure.

FIG. 5A depicts an example embodiment of a workout preview interface of a mobile application of a system for linking a product to product information according to the present disclosure.

FIG. 5B depicts an example embodiment of an exercise product information interface of a mobile application of a system for linking a product to product information according to the present disclosure.

FIG. 6 depicts an example embodiment of a workout summary interface of a mobile application of a system for linking a product to product information according to the present disclosure.

FIG. 7A depicts an example embodiment of a workout data entry interface of a mobile application of a system for linking a product to product information according to the present disclosure.

FIG. 7B depicts an example embodiment of a workout data display interface of a mobile application of a system for linking a product to product information according to the present disclosure.

FIGS. 8A and 8B depict example embodiments of an injury selection interface of a mobile application of a system for linking a product to product information according to the present disclosure.

FIGS. 9A-9C depict example embodiments of injury information and workout interfaces of a mobile application of a system for linking a product to product information according to the present disclosure.

FIG. 10 depicts an example embodiment of a recovery content and information interface of a mobile application of a system for linking a product to product information according to the present disclosure.

FIG. 11 illustrates an example flow diagram depicting acts associated with linking a product to product information, in accordance with implementations of the present disclosure.

### DETAILED DESCRIPTION

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning.

The method and system according to the present disclosure for linking a product to product information according to the disclosure may advantageously allow for a user to receive customized and accurate information regarding use of a product even without in-person training regarding the product.

Implementations of the present disclosure may facilitate various benefits over conventional techniques for providing information and/or additional functionality associated with physical products. For example, as discussed herein, at least some implementations of the present disclosure allow users to scan a scannable element (e.g., a QR code or barcode, or other scannable identifier) associated with a physical product (e.g., attached to or implemented into the physical product) with a user device (e.g., a scanning sensor associated with a mobile electronic device such as a smartphone, tablet, laptop, etc.). The scanning may cause the user device to navigate to one or more types of content related to the physical device or product.

In some instances, in response to scanning the scannable element, the user device is directed to information related to the use of the physical product associated with the scannable element. For example, where the physical product is an exercise apparatus, the content may comprise videos and/or step-by-step guides that direct users in the proper use of the exercise apparatus. In some instances, the format for displaying the content related to the use of the product may be intelligently selected using one or more machine learning modules or usage databases. For instance, a machine learning module or usage database may indicate a best format for presenting content related to the product based on various inputs, such as location of the user, age or demographics of the user, medical history of the user, biometric data of the user, health goals of the user, information about the device used by the user, a usage context (e.g., whether the user will perform an activity that precludes viewing of the user device), and/or other information. A selected format for content presentation may comprise, for instance, a step-by-step guide (e.g., in written form and/or with graphical elements), audiovisual guidance (e.g., video segments), real-time assisted guidance (e.g., via video and/or audio conference or streaming), audio-only guidance, video-only guidance, and/or others. The usage database and/or machine learning model may additionally or alternatively modify the content itself that becomes displayed for the user. For example, an elderly user who scans a dumbbell may be presented with different exercise options than a youthful person who scans a dumbbell.

In some instances, in response to scanning a scannable element, the user device is directed to a user prompt that prompts the user to provide input related to the user's intended use of the product and/or the user's desired result from using the product. For example, where the product associated with the scannable element is an exercise device, the user may be prompted to indicate whether they intend to use the exercise device for general fitness or for rehabilitative purposes (e.g., the user may indicate a particular type of injury that the user is experiencing or recovering from). The user device may then be directed to content related to the use of the product that is in accordance with the user input provided related to intended use and/or desired result.

In some instances, in response to scanning a scannable element, the user device is directed to a user interface for tracking usage of the product associated with the scannable element. For example, where the product associated with the scannable element is an exercise device, the user device may be directed to a user interface that allows the user to input information related to use of the exercise device (e.g., duration of workout, calories burned, distance covered, repetitions performed, time spent in particular positions, exercise activities/regimens completed, goals achieved, user biometric data exhibited during exercise such as heartrate, etc.). In other instances, functionality for tracking usage of the product may be activated in response to the scanning of the scannable element. For example, scanning the scannable element may trigger activation of one or more sensors for tracking the user's usage of the product. For instance, the scanning of the scannable element may trigger sensors of the user device (e.g., one or more cameras or image sensors, inertial measurement units, magnetometers, gyroscopes, accelerometers, microphones, barometers, thermometers, pedometers, and/or others) or associated with the user device (e.g., devices that communicate with the user device via a wireless connection, such as heartrate monitors, smartwatches or other fitness tracking watches, and/or others) to track attributes of the user's usage of the product (e.g., duration of workout, calories burned, distance covered, repetitions performed, time spent in particular positions, exercise activities/regimens completed, goals achieved, user biometric data exhibited during exercise such as heartrate, etc.). In some instances, the user device is directed to a user interface that displays statistical data associated with the user's historic usage of the product associated with the scannable element (or statistical data associated with other users' historic usage of the product associated with the scannable element).

The foregoing functionality may facilitate improvements to the efficiency and/or manner with which users access and/or update information related to products used by the users.

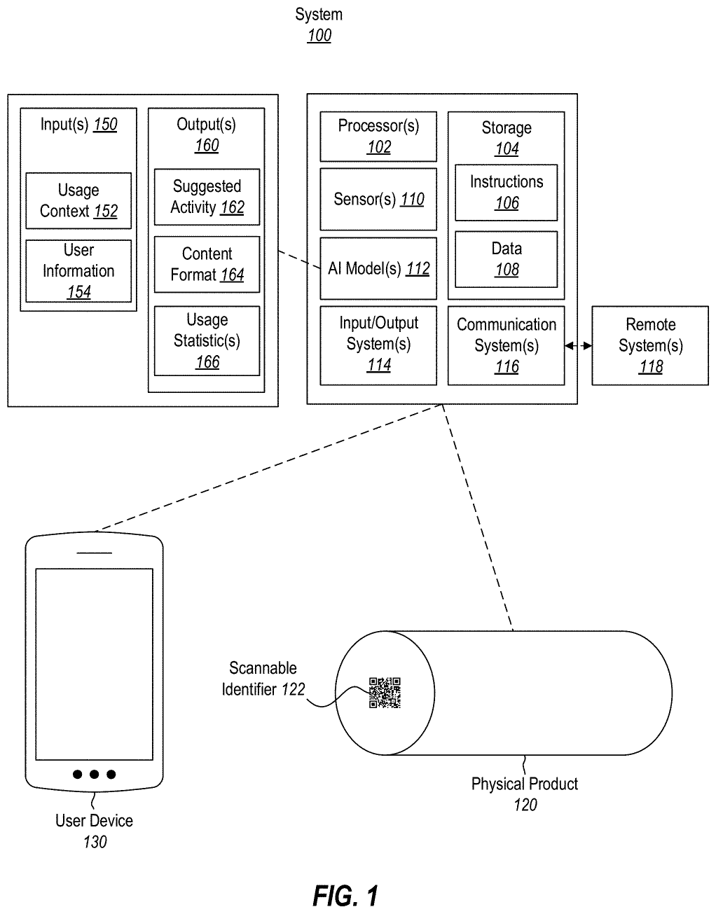

FIG. 1 illustrates various example components of a system 100 for facilitating linking of a product to product information, in accordance with the present disclosure. For example, FIG. 1 illustrates that a system 100 may include processor(s) 102, storage 104, sensor(s) 110, artificial intelligence model(s) 112 (AI model(s) 112), input/output system(s) 114 (I/O system(s) 114), and communication system(s) 116. Although FIG. 1 illustrates a system 100 as including particular components, one will appreciate, in view of the present disclosure, that a system 100 may comprise any number of additional or alternative components.

The processor(s) 102 may comprise one or more sets of electronic circuitries that include any number of logic units, registers, and/or control units to facilitate the execution of computer-readable instructions (e.g., instructions that form a computer program). Such computer-readable instructions may be stored within storage 104. The storage 104 may comprise physical system memory and may be volatile, non-volatile, or some combination thereof. Furthermore, storage 104 may comprise local storage, remote storage (e.g., accessible via communication system(s) 116 or otherwise), or some combination thereof. Additional details related to processors (e.g., processor(s) 102) and computer storage media (e.g., storage 104) will be provided hereinafter.

As will be described in more detail, the processor(s) 102 may be configured to execute instructions 106 stored within storage 104 to perform certain actions associated with linking a product to product information. The actions may rely at least in part on data 108 stored on storage 104 in a volatile or non-volatile manner.

In some instances, the actions may rely at least in part on communication system(s) 116 for receiving data and/or instructions from remote system(s) 118, which may include, for example, separate systems or computing devices, sensors, and/or others. The communications system(s) 118 may comprise any combination of software or hardware components that are operable to facilitate communication between on-system components/devices and/or with off-system components/devices. For example, the communications system(s) 118 may comprise ports, buses, or other physical connection apparatuses for communicating with other devices/components. Additionally, or alternatively, the communications system(s) 118 may comprise systems/components operable to communicate wirelessly with external systems and/or devices through any suitable communication channel(s), such as, by way of non-limiting example, Bluetooth, ultra-wideband, Wi-Fi, WLAN, infrared communication, and/or others.

FIG. 1 illustrates that a system 100 may comprise or be in communication with sensor(s) 110. Sensor(s) 110 may comprise any device for capturing or measuring data representative of perceivable phenomenon. By way of non-limiting example, the sensor(s) 110 may comprise one or more image sensors, optical scanners, microphones, thermometers, barometers, magnetometers, accelerometers, gyroscopes, pedometers, and/or others. As will be described in more detail hereinafter, sensor(s) 110 may include any image sensor and/or scanning device usable to detect the presence of a scannable identifier/element (e.g., a quick response (QR) code, barcode, or other structure).

Furthermore, FIG. 1 illustrates that a system 100 may comprise or be in communication with I/O system(s) 114. I/O system(s) 114 may include any type of input or output device such as, by way of non-limiting example, a display, a touch screen, a mouse, a keyboard, a controller, a speaker, a light source, a vibration motor, and/or others, without limitation. In some instances, I/O system(s) 114 may at least partially rely on the sensor(s) 110 (e.g., via a microphone or motion sensor system to detect user input).

FIG. 1 also depicts that one or more of the components of the system 100 may be implemented on a user device 130. For example, the processor(s) 102, sensor(s) 110, input/output system(s) 114, communication system(s) 116, and at least a portion of the storage 104 may be implemented on a user device 130 (e.g., a smartphone, tablet, personal computer, laptop computer, smartwatch, and/or another device). The user device 130 may communicate with one or more remote system(s) 118 (e.g., one or more servers, communication devices, etc.).

FIG. 1 illustrates that the system 100 may comprise or interact with a physical product 120. FIG. 1 further shows that a scannable identifier 122 may be associated with the physical product 120. FIG. 1 illustrates an example in which the physical product 120 is a foam roller usable for exercise, stretching, and/or rehabilitative purposes. FIG. 1 also illustrates the scannable identifier 122 as a QR code affixed to the foam roller. Notwithstanding the particulars of the example shown in FIG. 1, those skilled in the art will recognize, in view of the present disclosure, that a physical product 120 may comprise other types of objects, such as other types of exercise apparatuses (e.g., straps for facilitating user stretching, weightlifting machines/equipment, treadmills, rowing machines, elliptical machines, etc.) or other physical devices that are manipulable or controllable by users. Furthermore, the scannable identifier 122 may comprise any suitable form that is detectable by an optical sensor and/or image sensor, such as one or more barcodes, symbols, characters, colors, graphics, designs, structural features/elements, combinations thereof, and/or others. Still furthermore, the scannable identifier 122 may be associated with the physical product 120 in various ways. For example, the scannable identifier 122 may be integrated into the physical product 120 (e.g., the scannable identifier 122 may be formed from the material(s) that form the physical product), affixed to the physical product 120 (e.g., via one or more adhesives or other attachment features/mechanisms), or connected to or formed within one or more surfaces proximate to the physical product 120 (e.g., where the physical product 120 comprises a bench press, the scannable identifier 122 may be affixed to a floor, wall, column, or other structure element that is near the bench press).

As will be described in more detail hereinafter, the scannable identifier 122 associated with the physical product 120 may be scanned (e.g., using a scanning sensor of the sensor(s) 110 of a user device 130) to trigger display of various types of information related to the physical product 120.

FIG. 1 illustrates that a system 100 may comprise AI model(s) 112 (e.g., accessible to a user device 130, whether stored on the user device 130, remote system(s) 118, or some combination thereof). The AI model(s) 112 may comprise or be configurable to execute any combination of software and/or hardware components that are operable to facilitate processing using machine learning models or other artificial intelligence-based structures/architectures. For example, one or more processors may comprise and/or utilize hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

The AI model(s) 112 may be configurable to generate various output based on various input. For instance, FIG. 1 depicts the AI model(s) 112 associated with various input(s) 150 and output(s) 160. According to the example shown in FIG. 1, the input(s) 150 may include usage context 152, user information 154, and/or others. Furthermore, according to the example shown in FIG. 1, the output(s) 160 may include suggested activity 162, content format 164, usage statistic(s) 166, and/or others.

For instance, after having scanned the scannable identifier 122 with a sensor of a user device 130, the AI model(s) 112 may be accessed to facilitate presentation of information related to the product 120 that is associated with the scannable identifier 122. Input for the AI model(s) 112 may be provided to generate output using the AI model(s) 112, and the output may determine aspects of information about the product 120 for presentation on the user device 130.

By way of non-limiting example, where the physical product 120 is an exercise apparatus, the usage context 152 may indicate a time of day, schedule, location, sequence of scanning relative to the scanning of scannable identifiers of other products, a mode or state of the user device 130 or other aspect of the system 100, and/or other contextual information related to the physical product 120 and/or the scanning of the scannable identifier 122 thereof. By way of further illustration, where the physical product 120 is an exercise apparatus, the user information 154 may comprise user age, user race, user ethnicity, user national origin, sex/gender of the user, exercise goals or desired outcomes of the user, medical/condition (e.g., past or present injuries, existence of a pacemaker or other implanted devices, etc.), and/or other information related to the user.

Based on the input(s) 150 (e.g., usage context 152 and/or user information 154), the AI model(s) 112 may be configured or trained to generate various output(s) 160 (e.g., using training data comprising (i) training inputs of usage context and/or user information and (ii) ground truth output of suggested activity, content format, and/or usage statistics) that inform the product information displayed in response to scanning of a scannable identifier 122 associated with a physical product 120.

By way of non-limiting example, where the physical product 120 is an exercise apparatus, the suggested activity 162 may comprise one or more exercise activities and/or exercise regimens performable using the exercise apparatus. In some instances, where the suggested activity 162 includes one or more exercise activities and/or exercise regimens performable using an exercise apparatus, the content format 164 may comprise one or more formats through which exercise activities and/or exercise regimens may be conveyed to users (e.g., a textual format, a still graphics format, a video format, an audio format, and/or an audiovisual format). Furthermore, where the product 120 is an exercise apparatus, the usage statistic(s) 166 may relate to one or more users' use of the exercise apparatus (e.g., a user operating the user device 130 and/or other users).

As indicated above, the various example output(s) 160 discussed above may be intelligently determined based on the input(s) 150 discussed above (e.g., via AI model(s) 112 and/or a usage database). By way of illustrative example, a geriatric user with an anterior cruciate ligament (ACL) injury may scan a scannable identifier 122 associated with an exercise strap using a user device 130 while located at a gymnasium. In response to the scanning, the user device 130 (and/or systems associated therewith, such as one or more servers) may obtain usage context 152 data and/or user information 154. It will be appreciated that the usage context 152 and/or the user information 154 may be obtained in various ways, such as by prompting a user to input such information, obtaining sensor data indicating such information, accessing one or more data structures where such information is stored/accessible, etc.

Continuing with the foregoing example, the user device 130 may access a user profile associated with the user to determine at least that the user is a geriatric user, access location data associated with the user device 130 to determine that the user is at a gymnasium, and/or provide a user prompt to the user to allow the user to indicate that they would like to use the scanned exercise strap for rehabilitative purposes to recover from an ACL injury (as opposed to desiring to use the exercise strap for other health/fitness purposes). The foregoing usage context 152 and/or user information 154 may be utilized as input(s) 150 to AI model(s) 112 to determine appropriate content to provide to the user of the user device 130. For instance, based on the foregoing input(s) 150 of the present example, the AI model 112 may output a set of ACL rehabilitation exercises that are performable using the scanned exercise strap and that are suitable for geriatric users (suggested activity 162), rather than other exercise strap activities that are not tailored to ACL rehabilitation and/or are not well-suited for geriatric users. The set of exercise strap ACL rehabilitation exercises may be further optimized to include exercise strap exercises that interdepend on other equipment that is available at the gym in which the scannable identifier 122 of the exercise strap was scanned (or to omit exercises that interdepend on equipment that is likely unavailable).

Further to the foregoing example, the set of exercise strap ACL rehabilitation exercises may be provided or presented in a format that is selected based on usage context 152 and/or user information 154. For instance, based on determining that the user is in a noisy environment with no available headphones, the set of exercises may be provided in a text, graphical, or video format, whereas in other instances, based on determining that the user device has limited display functionality (e.g., where the user device is a watch), the set of exercises may be provided in an audio format.

In addition, or as an alternative, to providing a set of exercise strap ACL rehabilitation exercises, the output(s) 160 of the AI model(s) 112 may comprise usage statistic(s) 168, which may assist the user in forming expectations for their performance while using the exercise strap (e.g., for the intended purpose of ACL rehabilitation, according to the present example). For instance, based on at least some of the input(s) 150 discussed above, usage statistic(s) 166 related to other geriatric users' historic use of similar exercise straps for ACL rehabilitation may be displayed to the user (e.g., a number of repetitions that the user should expect to be able to perform for certain exercises, ranges of motion that the user should expect to be able to achieve at various points through an exercise plan, etc.). In some instances, instead of providing usage statistic(s) 166 associated with other users, scanning the scannable identifier 122 of the physical product 120 triggers display of usage statistics based on the user's own past use of the physical product 120 (or similar physical products).

In this way, the product information provided in response to scanning the scannable identifier 122 associated with the exercise strap may be tailored (in form and/or content) to the particular user and/or the particular usage context relevant to the user at the time of scanning. One will appreciate that the foregoing example is illustrative only and not limiting of the present disclosure.

Additional or alternative functionality may be facilitated by system 100 in response to scanning a scannable identifier 122 of a physical product, and not all functionality described herein need rely on AI model(s) 112. For instance, in some implementations, scanning a scannable identifier 122 may cause a user device 130 to navigate to a user interface that allows the user to input data related to the user's current or recent use of the physical product 120 associated with the scannable identifier 122 or that allows the user to view data related to the user's historic use of the physical product 120 associated with the scannable identifier 122. In some implementations, scanning a scannable identifier 122 associated with a physical product 120 causes the system 100 to activate sensor(s) 110 for tracking the user's usage of the physical product 120. For instance, where the physical product 120 comprises a treadmill or elliptical machine, scanning a scannable identifier 122 associated with the treadmill or elliptical machine may activate (or queue for activation) one or more inertial measurement units and/or other sensors to facilitate tracking of the user's steps while using the treadmill or elliptical machine.

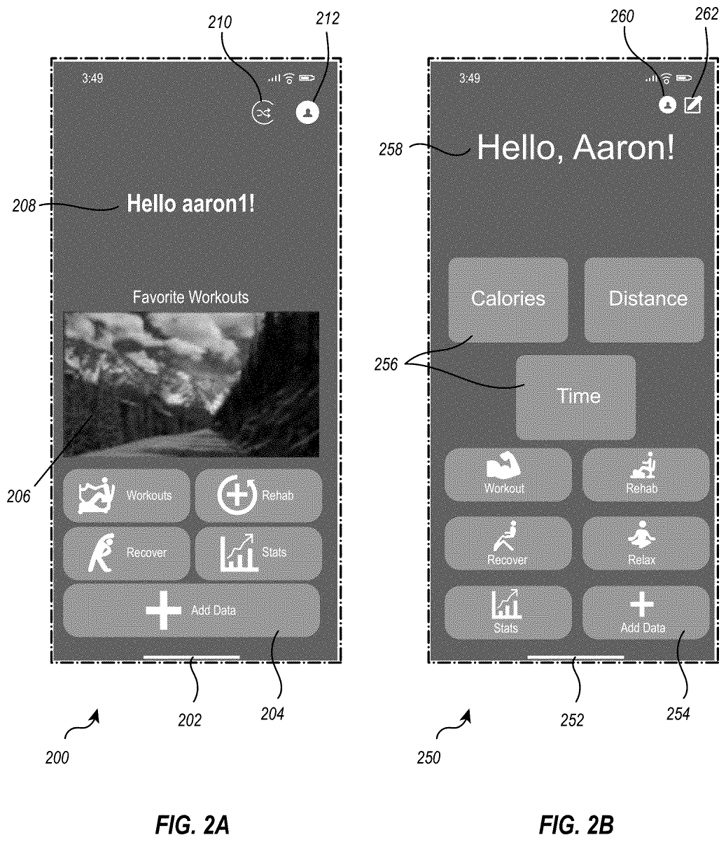

As noted herein, a system 100 may include one or more input/output system(s) 114, at least some of which may be configured as displays for displaying content to users. In some instances, such features may be implemented as a screen of a user device 130, such as a smartphone screen configured to display various user interfaces to users. FIGS. 2A-10 illustrate various example user interfaces that may be displayed to users in accordance with implementations of the present disclosure.

FIG. 2A shows a home page of a mobile application of a system according to an embodiment of the present disclosure. A mobile application 200 may comprise a user interface 202. The user interface 202 may comprise one or more buttons or options 204 corresponding to features of the mobile application 200. For example, the buttons or options 204 may allow a user to navigate to a section such a workouts section, a rehabilitation section, a recover section, a statistics section, or a data input section. It will be understood that the depicted embodiment is merely provided by way of example, and any suitable combination of features may be utilized.

A featured section 206 may be displayed in at least a portion of the user interface 202. In embodiments, the featured section 206 may display a favorites library, where a user's preferred or most frequently selected features are stored and made easily accessible by the user. In the user interface 202, the featured section 206 presents the user's favorite workouts based on previous use and the user's purchased products which correspond to particular workouts. For example, the user interface 202 may correspond to a mobile application for a vendor of medical or physical-therapy products, and the favorite workouts pertain to use of a particular medical or physical-therapy product.

The user interface 202 may further comprise a messaging component 208, which in embodiments may display a welcome message as shown in FIG. 2A to a user. The messaging component 208 may be customized, and a welcome message or other message displayed thereon may be specified by a user or determined based on previous messages, engagement therewith, or other users' engagement with various messages using a machine learning model as described herein.

The user interface 202 may further comprise a menu button 210 and/or a profile/account button 212. The menu button 210, when clicked, may allow a user to navigate a menu of features of the mobile application. The profile/account button 212, when clicked, may allow a user to update account or personal information and preferences in the application 200.

An alternative embodiment of a mobile application cooperating as part of a system and method for linking a product to product information is depicted in FIG. 2B. The mobile application 250 may comprise a user interface 252 that comprises one or more buttons or options 254 that are linked to features of the mobile application. For example, the features may include a workout section, a rehabilitation section, a recovery section, a relaxation section, a statistics section, and/or a data input section.

The user interface 252 may further comprise one or more indicia 256 corresponding to the user's progress or compliance with one or more goals or metrics. The indicia 256 may define a shape or symbol and may comprise lettering that indicates what the indicia pertains to, for example calories, distance, or time as shown in FIG. 2B.

As described herein, a user may adopt one or more predetermined goals or metrics from the mobile application 250 pertaining to a number of calories to be burned in a predetermined time period such as a 24-hour period, a distance equivalent to cover in a predetermined time period, and/or a duration of exercise to complete within a predetermined time period.

For example, a default metric may be provided in the mobile application 250, which, if selected or adopted by a user, measures the user's progress towards a 2,500-calorie goal, a 5.0 mile equivalent goal, and/or a 1:00 of activity goal for each day. The user's progress towards each of these metrics as measured by the user's input data and/or tracked data using the mobile application 250 may be assessed against the goal, with a color chosen for each indicium of the one or more indicia 256 showing the user their progress generally.

The indicia 256 may be updated or controlled according to a machine learning model which may track the user's progress according to one or more different input metrics. For example, the mobile application 250 may track the user's activity based on the internal sensor information of their mobile device (e.g., the on-board compass, accelerometer, gyroscope, or other sensors, or using the mobile device's location as determined by a GPS modality, cellular data, Wi-Fi, barometer, or Bluetooth, for example) to determine distance travelled on foot, number of steps, etc.

The machine learning model may further account for the user's engagement with the app, such as a user completing a workout video. This may further be combined with data obtained from paired devices, such as smart watches and fitness trackers. The machine learning model may further base a determination on a user's past behaviors, such as whether a user viewing a workout video through the app was accompanied by data from a paired device such as a Fitbit indicating the degree to which the user was following along and exercising along with the video instructor.

In embodiments, a user may specify one or more goals or metrics using one or more of the data input section or the profile/account section. The user may select from a plurality of metrics not limited to calories, distance, and duration of exercise. The user may additionally select from, for example, a total step goal, an hourly step goal, a mindfulness duration goal, a sleep duration goal, a water consumed goal, a fruits and/or vegetables servings consumed goal, a variety of exercise goal, or any other suitable goal. The metrics or goals may further serve to indicate whether a user has limited a certain goal, such as staying below a daily number of calories consumed, cigarettes smoked, unhealthy drinks or snacks consumed, or time spent sitting or spent on a particular mobile application or website, for example.

Although no color is shown in FIG. 2B, colors for each indicium of the indicia 256 can be used to show progressing (e.g., from red (indicating poor or insufficient compliance) to yellow (indicating partial compliance) to green (indicating compliance)). Any suitable combination or spectrum of colors, symbols, sizes, and configurations of the indicia 256 may be utilized. For example, in embodiments the one or more indicia may be provided in an ordered list, showing the metric or goal which requires most attention in order to achieve compliance at the top of the list. Alternatively, the metric or goal which requires most attention to achieve compliance may be shown larger than the other indicia and/or may flash, pulsate, or otherwise gain the user's attention when using the mobile application 250.

The user interface 252 may further comprise a messaging component 258, which in embodiments may display a welcome message as shown in FIG. 2B to a user. In other embodiments the messaging component 258 may prompt a user to focus on one or more of the goals or metrics represented by the indicia 256. For instance, the messaging component 258 may state “20 more minutes of cardio needed today!” or “Great job meeting your calories goal!”.

The user interface 252 may comprise a profile/account button 260, and/or a messaging button 262 allowing the user to communicate with other users of the mobile application 250 or with representatives of the vendor.

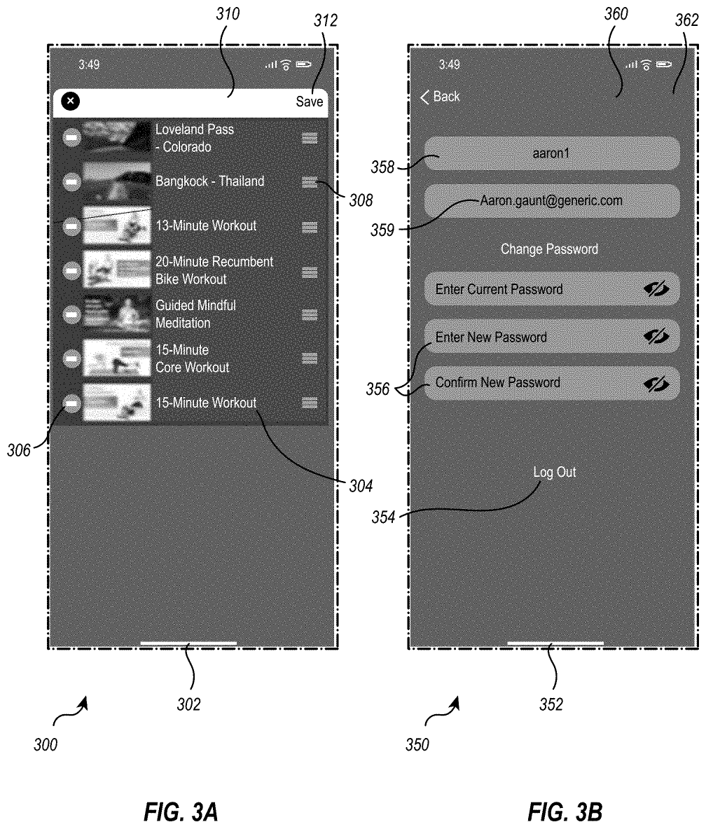

Turning to FIGS. 3A-3B, another user interface corresponding to a mobile application according to embodiments is shown. A mobile application 300 according to embodiments may comprise a user interface 302 corresponding to a favorites feature or a featured section as described herein. The featured section of the interface 302 may be configured to allow a user to arrange their favorite content, such as workout videos or instructions, in a desired configuration.

For example, a user may select which content to display in the featured section of the home page as shown in FIG. 2A. The user interface 302 may show a plurality of information or content 304 from which the user may select favorite content for the featured section. The information or content 304 from which the user may select may be provided based on the user's history, including products, videos, and/or workouts previously viewed, and/or may be determined by the machine learning model based on predictions of videos or workouts that the user may appreciate given previous engagement.

The user interface 302 may provide a remove button 306 allowing a user to remove a suggested content from the featured section, and/or a menu button 308. In embodiments, the menu button 308 may be used by the user to adjust an order or ranking of the content 304, such that by pressing the menu button 308, the user may drag or otherwise move the content 304 up or down relative to other content. This may advantageously allow the featured section to display content in precisely the order that a user wishes to see.

In the embodiment of FIG. 3B, a mobile application 350 may comprise a user interface 352 corresponding to a profile/account button as described regarding the foregoing embodiments. The profile/account button, when clicked, may navigate the user to the user interface 352 defining a profile section as shown in FIG. 3B. The profile section may display a user's information, including a username 358 and an email address 359 associated with the user's account. The profile section may further facilitate the user changing their password using the password fields 356. For example, the user may be required to enter the current password in one of the password fields 356, enter the new password in a second one of the password fields 356, and finally confirm the new password in a third one of the password fields 356. The user interface 352 may further comprise a log out button 354 allowing the user to log out of their account on the mobile application 350.

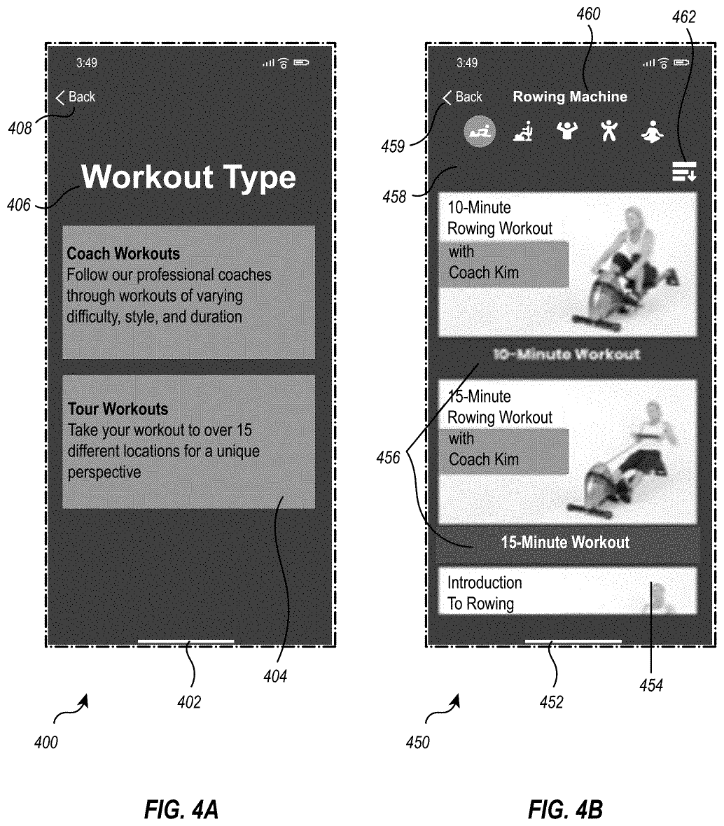

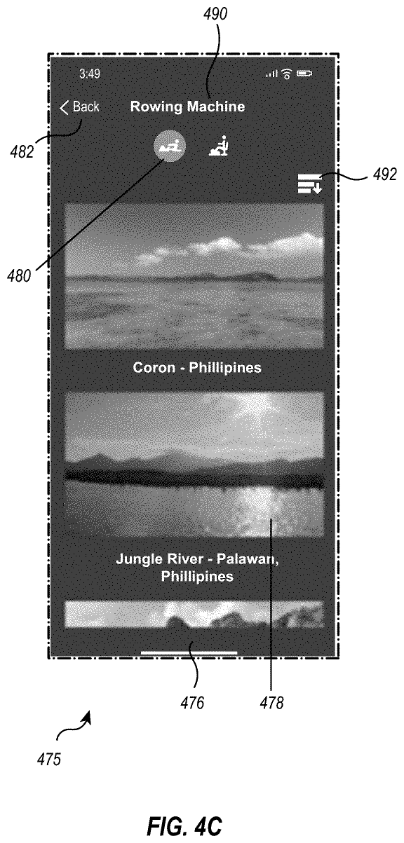

Turning to the embodiment of FIGS. 4A-4C, a mobile application 400 according to a system for linking a product to product information according to the present disclosure, may comprise a workout-type selection section 402 defining a workout-type selection section. The workout-type selection section 402 may facilitate selection between different categories of workouts. In the depicted embodiment, the categories include coach workouts (in which a professional coach such as a personal trainer or physical therapist leads and/or demonstrates a workout) and tour workouts (in which different locations are virtually presented and traveled through during the course of the workout). The workout-type selection section 402 may comprise a menu header 406 indicating the selection that is available, and a back button 408 enabling a user to navigate back to a home page.

The embodiment of FIG. 4B indicates the different types of content that may be accessed by clicking on one of the options available from the user interface of FIG. 4A, in particular the coach workouts option. In embodiments, a mobile application 450 may comprise a user interface defining a workout selection section 452. The workout selection section 452 comprises one or more selectable workouts or content 454. The content 454 may be grouped into groups 456 according to any suitable categorization, including duration of workout, such as groups of 5-minute, 10-minute, and 15-minute workouts, respectively. Other categorizations may include focus on particular muscle groups, aerobic vs. strength training, warm up vs. cool down, degree of difficulty (e.g. easy, medium, hard), or any other suitable categorization.

The workout selection section 452 further comprises one or more different types of exercise that may be switched and selected between. For example, one or more buttons 458 may be selected to show a different menu or selection of content. In the embodiment of FIG. 4B, the buttons 458 take the form of an icon showing the type of workouts or content corresponding to the button. The buttons 458 may include, for example, rowing machine, recumbent bike, strength training, calisthenics, and mindfulness, respectively. Any other suitable category may be used, and the above selections are merely exemplary. Once a button 458 is selected, the name of the corresponding library of content may be shown at bar 460.

A menu button 462 may allow a user to sort the content of the selected button 458 in any suitable manner, for instance according to different categories as described above. The user may organize the workouts according to a particular coach demonstrating the workout in the video, according to a difficulty level of the workout, by style of workout such as stamina, sprint, warm-up, or cool-down, or by prioritizing workouts that have not been completed by the user previously, for example. The workout selection section 452 may further comprise a back button 459 to allow the user to return to the workout-type selection section 402.

The embodiment of FIG. 4C illustrates a workout-tour selection section. In the mobile application embodiment 475 of FIG. 4C, a user interface defines a workout-tour selection section 476 to which a user may be navigated after selecting the tour workouts button 404 of the workout-type selection section 402. The workout-tour selection section 476 may include a plurality of content 478 that may be selected by a user.

For example, a user may select from a plurality of locations through which to virtually travel while performing an exercise routine. The locations may correspond to the user's selection of a type of exercise. For example, buttons 480 may allow a user to select a different menu or selection of content that corresponds to a rowing machine or an elliptical machine, as shown in FIG. 4C.

Thus, the locations for the rowing machine may include water-based settings, while the locations for the elliptical machine may comprise mountainous settings. While locations corresponding to a rowing machine-based workout content library and an elliptical machine-based workout content library are described in regards to FIG. 4C, it will be appreciated that any suitable type of location and any suitable number of locations may be provided for any suitable type of exercise as suitable. A bar 490 may show the name of the content library corresponding to a selected button 480.

As with the embodiment of FIG. 4B showing a menu button 462, a menu button 492 may allow a user to toggle between workout content in the workout-tour selection section 476 based on any suitable categorization of the content. In embodiments, the user may use the menu button 492 to categorize references based on a duration of the workout, a geographical location, a difficulty level, or otherwise. The workout-tour selection section 476 may comprise a back button 482 allowing a user to return to the workout-type selection section 402.

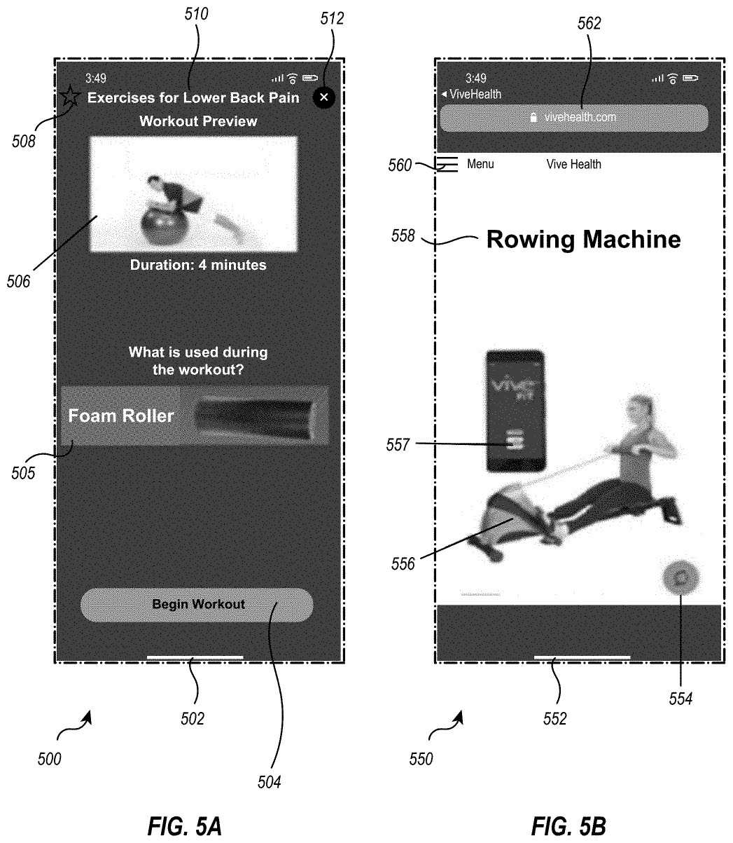

According to the embodiments of FIGS. 5A and 5B, a mobile application 500 may comprise a user interface defining a workout preview with store redirection section 502. The workout preview with store redirection section 502 advantageously comprises a begin workout button 504 allowing a user to initiate a workout instruction, such as a video-directed workout. The workout preview with store redirection section 502 may further comprise an indicium 505 providing information about a product available from the vendor suitable for the selected workout.

In embodiments, the product is a medical device such as a foam roller that the user may have purchased and received from the vendor. The mobile application 500 may populate the indicium 505 and navigate the user to workouts compatible with the product (e.g., suggested activity 162, as discussed hereinabove with reference to FIG. 1), upon the user scanning an identifier such as a QR code (e.g., scannable identifier 122, as discussed hereinabove with reference to FIG. 1) provided on the product packaging (or on the device itself, such as physical product 120, as discussed hereinabove with reference to FIG. 1). In embodiments, the indicium 505 is a button that, when clicked or pressed by the user, navigates the user to the vendor's website for further information and/or to order the product.

The workout preview with store redirection section 502 may further comprise a workout preview 506 comprising indicia regarding a duration of the workout and an image or video of the workout to be performed. The workout preview 506 may show a clip of a video of the workout in a continuous loop. The workout preview 506 may be shown upon a user selecting the workout from a menu or library of content. For example, the workout shown in FIG. 5A may be selected from a library 510 titled Exercises for Lower Back Pain. A button 508 allows a user to save or bookmark the library 510 for future ease of access. In embodiments, the button 508 allows the user to save the particular workout for future ease of access. A button 512 allows a user to exit the selected workout and return to the library 510 or to a home page (e.g., workout-type selection section 402).

FIG. 5B shows an embodiment of a mobile application or website 550 to which the user may be navigated upon clicking on the indicium 505 regarding the product corresponding to a particular workout. The mobile application or website 550 may comprise a user interface configured as a virtual storefront 552. The virtual storefront 552 may comprise a button 554 allowing the user to purchase the product. Indicia 556 may show the product from one or more different views, and the user may swipe between different figures of the product. A button 557 may be configured to navigate the user back to the application and corresponding library content pertaining to the particular product.

The title and other details regarding the product may be displayed regarding indicia 558. The virtual storefront 552 may comprise a menu button 560 allowing the user to navigate back to other categories of products as desired. The address of the virtual storefront 552 may be shown at bar 562 and may be edited thereat.

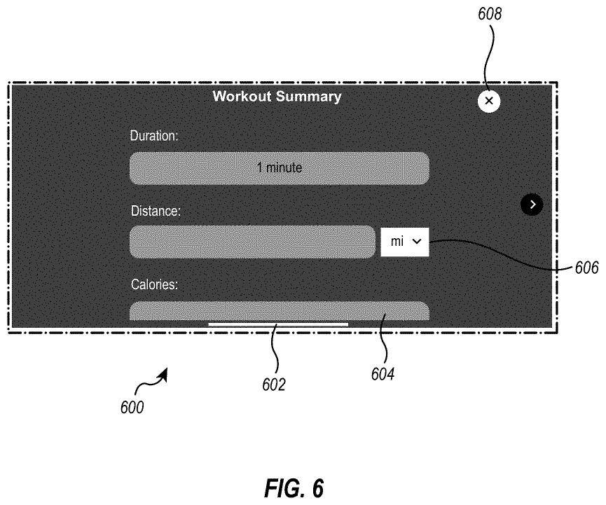

FIG. 6 illustrates an embodiment of a mobile application according to the present disclosure. The mobile application 600 may comprise a user interface 602 configured as a pop-up that can be provided to a user before, during, or after a workout as described herein. For example, a user may scan a scannable identifier 122 of an exercise device (physical product 120) that the user will use, is using, or has used to perform an exercise activity. In response to the scanning, the mobile application 600 may display user interface 602 with input fields to allow the user to enter metrics related to the user's performance of the exercise activity using the exercise device.

The pop-up user interface 602 may prompt the user to enter one or more fields 604 of information regarding the workout, such as a duration, a distance, and/or calories expended. The units of measure for one or more of the fields 604 may be toggled at button 606. Other exercise metrics not explicitly shown in FIG. 6 may be implemented, such as repetitions performed, time spent in one or more positions, exercise activities/regimens completed, goals achieved, biometric data exhibited during exercise, and/or others.

A button 608 allowing the user to close out of the pop-up user interface 602 may be provided. The information provided in the fields 604 may enable the machine learning model to customize the mobile application to the user. For example, it may be determined that the user does not tend to finish certain types of workouts, while other types of workouts and/or products correlate to the user finishing the workout more consistently and/or achieving more calories, distance, or duration during the workout. The information provided may be populated in the statistics section of the application.

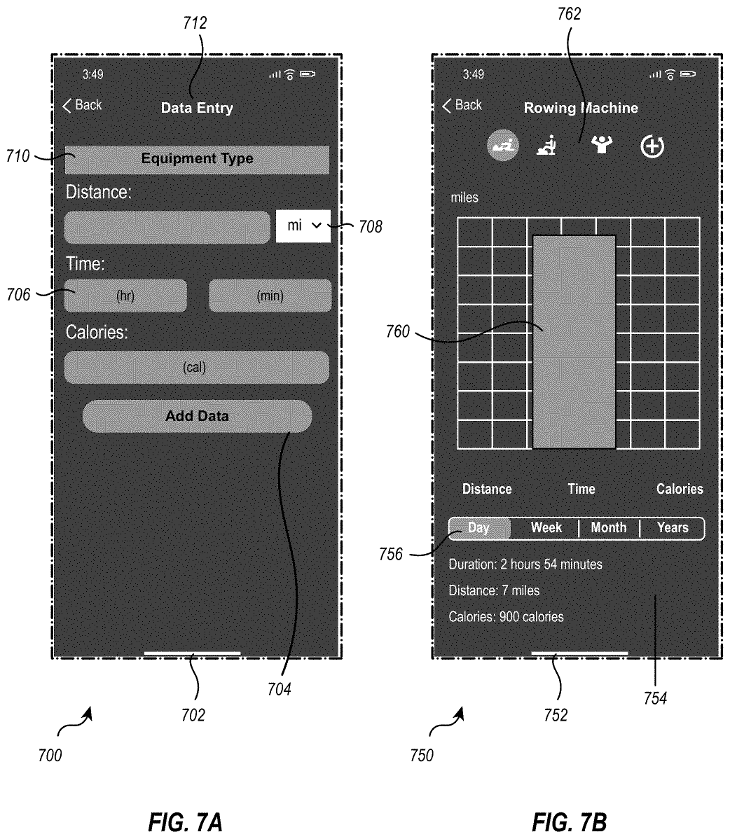

Another aspect of the present disclosure is described regarding FIGS. 7A and 7B. The application may include a data input section as described previously. A mobile application 700 may comprise a user interface defining a data input section 702. The data input section 702 may provide one or more fields 706 in which information regarding a workout, including distance, calories, and duration, may be manually entered by the user. A button 708 may allow the user to toggle the units of measure of the information in one or more of the fields 706.

A button 710 may allow a user to specify a type of exercise equipment on which the workout was completed. In embodiments, the button 710 allows the user to select a product with which the workout was completed. In some instances, the equipment type is automatically determined based on the scanning of a scannable identifier 122 associated with a physical product 120. Once the user has added the desired data, the user may click or select a button 704 to enter the data, which may populate the statistics section of the application and facilitate the machine learning model to further customize and improve the application based on the user's preferences and patterns of behavior. For instance, based on the metrics entered related to a user's performance of various types of exercises, output of a machine learning model (e.g., AI model(s) 112) may configure a user device (e.g., user device 130) to provide messages or other types of output to encourage the user as the user attempts to complete an exercise activity, regimen, or objective that the user struggles with completing or accomplishing (e.g., as determined based on the input related to the user's performance of exercise activities).

Once the data has been entered in the data input section 702, the data may be displayed and viewed in a user interface as shown regarding FIG. 7B. A mobile application 750 according to embodiments may comprise a user interface defining a statistics page 752. The statistics page 752 may be configured to be shown automatically to the user following data input using the data input section 702. The statistics page 752 may comprise a bar graph 760 showing metrics regarding one or more of the fields 706 from the data input section 702. The bar graph 760 may be toggled between different fields 706 using buttons 758, which each correspond to the fields 706.

Additionally, buttons 756 allow a user to switch between different time frames to review the input data. For example, a user may see total distance for a day, week, month, or year by toggling the buttons 756 and 758. A summary 754 is provided with all of the pertinent workout-related metrics in the statistics page 752. The statistics page 752 may further show information gathered from workouts performed on different workout equipment, which may be selected using the buttons 762. The buttons 762 may in embodiments correspond to whether the user is in a recovery stage or a rehab stage.

For example, a user may have completed workouts on a plurality of pieces of workout equipment or using a plurality of types of exercise, which can be selected by clicking on the corresponding button 762. As described previously, the bar graph 760 may have any suitable color, and in embodiments may have a color corresponding to the user's progress in attaining a predetermined metric or goal, i.e., showing the bar graph as green when the user's workout has satisfied the predetermined metric regarding the selected field. In some instances, a system navigates to a statistics page 752 directly in response to scanning of a scannable identifier 122 of a physical product 120 (e.g., an exercise device) to trigger display of performance statistics for a user associated with the user device that scans the scannable identifier 122 (or to trigger display of performance statistics for other users to allow the user to compare himself/herself to the other users).

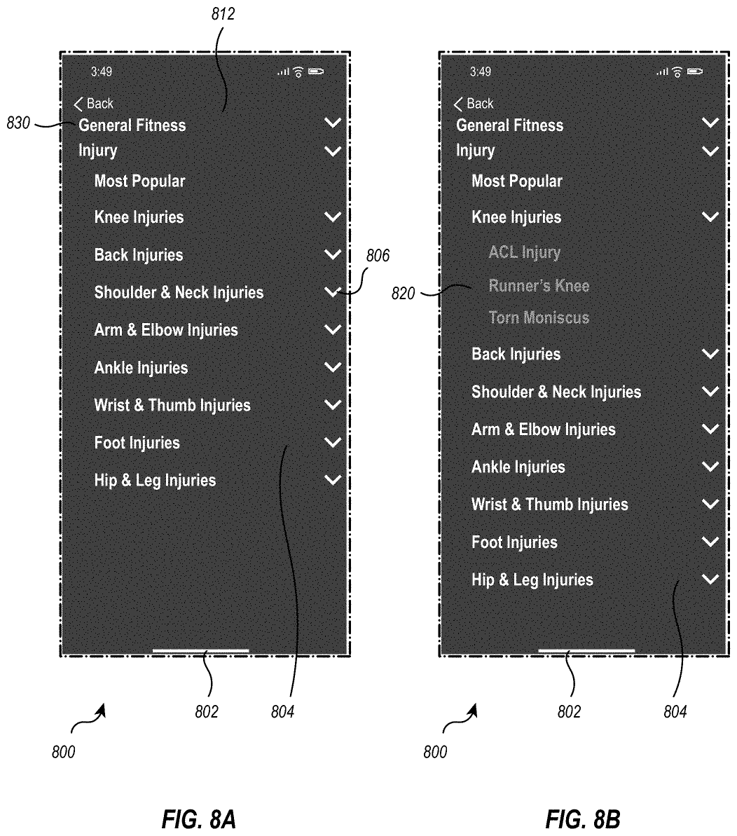

Turning now to FIGS. 8A and 8B, a mobile application 800 according to the disclosure may comprise a menu of content particularly geared to rehabilitation. The mobile application 800 comprises a user interface defining a rehab menu 802. The rehab menu 802 may display one or more fields 804 which may be selected by a user, the fields 804 corresponding to categories of injuries. The categories may be organized in any suitable manner and are described regarding the rehab menu 802 as being grouped by body part, such as knee injuries, back injuries, etc. As discussed above, a rehab menu 802 (or a similar user interface) may be displayed on a user device 130 in response to the user device scanning a scannable identifier 122 associated with a physical product. For example, where the physical product 120 is an exercise band or strap usable to carry out various rehabilitative exercises for overcoming injuries to various parts of a user body, scanning a scannable identifier 122 associated with the physical product 120 may trigger display of a rehab menu 802 that allows the user to specify a particular injury that the physical product 120 can be used to facilitate rehabilitative exercises for. In contrast, if the user would like to simply use the scanned physical product 120 for general fitness, the user may select a general fitness icon 830.

In the example shown in FIGS. 8A and 8B, the user may click a drop-down tab 806 of a desired category to drill down to further levels of categorization of the library content. The fields 804 may include other categories, such as most popular, recently viewed, and other categories based on customization of the application 800 for the user. FIG. 8B shows the rehab menu 802 when the user clicks the drop-down tab 806, showing further subcategories 820 that allow a user to conveniently navigate to a desired library of content.

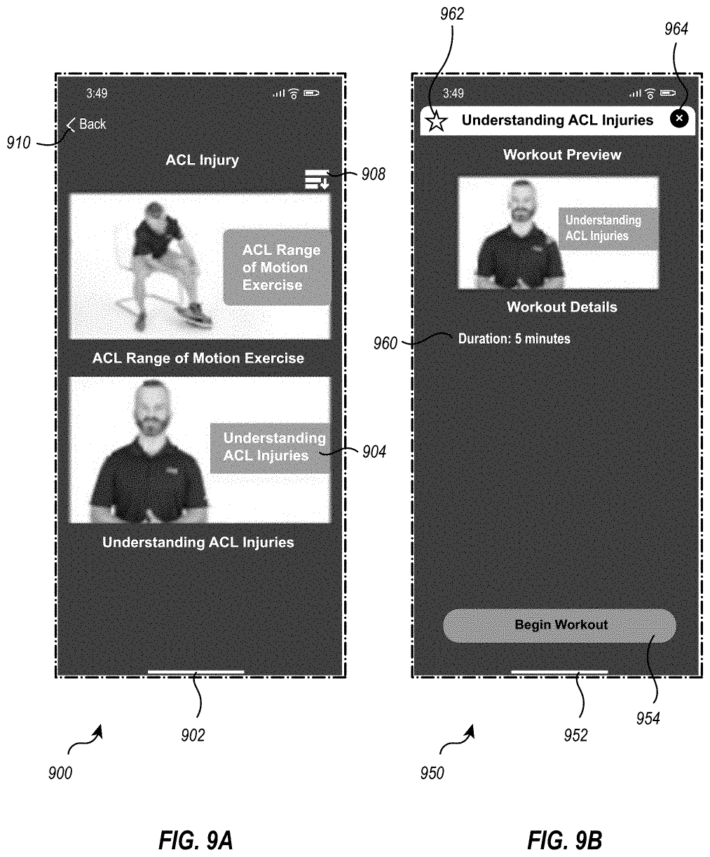

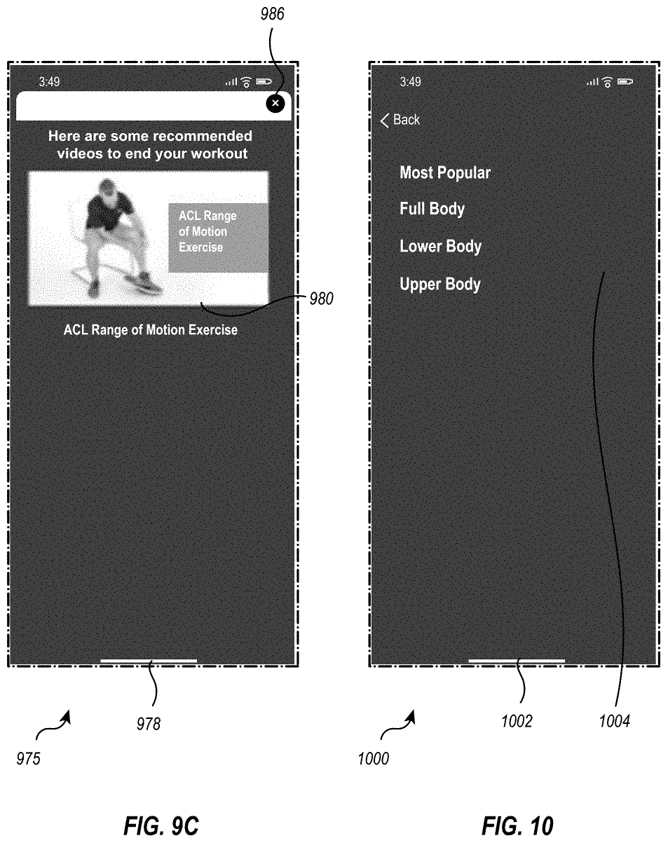

FIGS. 9A-9C illustrate an embodiment in which a user selects a particular injury type from a rehab menu 802 as described previously. The embodiment of a mobile application 900 may include a user interface 902 in which one or more individual content or information items 904 may be provided to the user. However, it will be appreciated, in view of the present disclosure, that a mobile application 900 (e.g., of a user device 130) may navigate to content similar to that shown in FIGS. 9A-9C without a user first explicitly selecting an injury type from a rehab menu 802 as previously described. For example, in some instances, a user scans a scannable identifier 122 associated with a physical product, and the scanning causes input related to the user (e.g., known attributes of the user, such as the user's current injuries obtained from a user profile that tracks the health status of the user) to be provided to an AI model that generates output that is usable to determine appropriate exercises and/or information to display to the user on the user device. Such exercises and/or information may be displayed to the user without the user first explicitly selecting a particular injury type from a rehab menu surfaced in response to scanning of a scannable identifier.

The content or information items 904 may be videos in which a physical therapist explains and demonstrates exercises pertaining to the selected injury, such as an anterior cruciate ligament (ACL) injury. A back button 910 allows the user to return to the rehab library to parse other injuries or other content as desired. A menu button 908 allows the user to more easily sort through the library according to desired categories or sorting options, such as sorting the content that has not been previously viewed.

A mobile application 950 including a user interface 952 may be presented to a user upon selection of one of the content or information items 904 from the user interface 902. The user interface 952 may define a rehab injury preview and workout summary. The user interface 902 may provide a button 954 allowing the user to initiate the workout, an indicium 960 showing information previewing details of the workout, and a bookmark option 962 and a close-out option 964.

A mobile application 975 including a user interface 978 may be presented to the user upon completion of the content or information items 904. The user interface 978 may display one or more content or information items 980 to end the workout, such as a demonstrate of exercises for further rehabilitating an injured body part. The user interface 978 may comprise a close-out button 986 to end the workout and return to a home page or a previous library. The information items 980 may be manually selected, i.e., predetermined, or may be supplied by the machine learning model based on the user's engagement with the application.

A mobile application 1000 as shown in FIG. 10 may comprise a user interface 1002 directed to recovery content and information. The user interface 1002 may define a menu of categories 1004 which the user can select, and as described regarding other embodiments, may provide targeted content or information items to a user based on a body part, such as a knee, that required recovery exercises and information, and which may be used in conjunction with a product from the vendor.

The following discussion now refers to a number of methods and method acts that may be performed by the disclosed systems. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

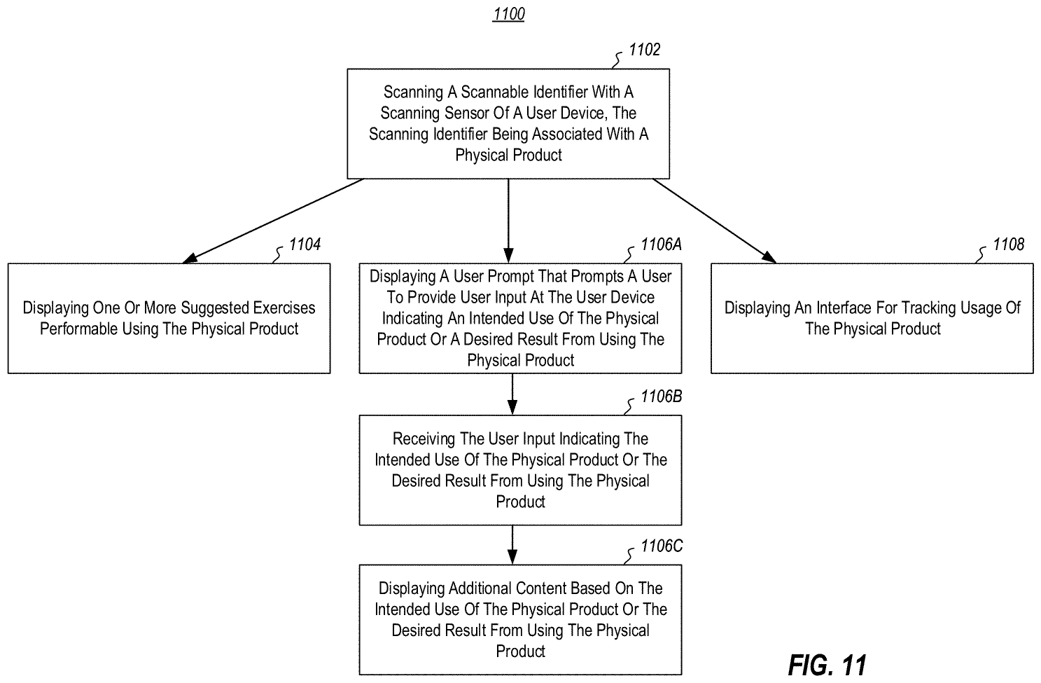

FIG. 11 illustrates an example flow diagram 1100 depicting acts associated with linking a product to product information. The discussion of the various acts represented in the flow diagram 1100 includes reference to various hardware components described in more detail with reference to FIG. 1.

Act 1102 of flow diagram 1100 includes scanning a scannable identifier with a scanning sensor of a user device, the scanning identifier being associated with a physical product. Act 1102 is performed, in some instances, by a system 100 utilizing processor(s) 102, storage 104, sensor(s) 110, input/output system(s) 114, communication system(s) 116, and/or other components. In some implementations, the scannable identifier is affixed to the physical product or integrated into the physical product. In some instances, the scannable identifier comprises a quick response (QR) code. Furthermore, in some instances, the physical product comprises an exercise apparatus.

The scannable identifier can be accessible to the user, and the scannable identifier may be configured to cause a user device to navigate to content related to use of the physical product in response to scanning of the scannable identifier with a scanning sensor associated with the user device. As is evident from FIG. 11, various different content may be displayed in response to the scanning of the scannable identifier, as indicated in FIG. 11 by acts 1104, 1106A, and 1108 all stemming from the scanning of the scannable identifier in accordance with act 1102.

Act 1104 of flow diagram 1100 includes displaying one or more suggested exercises performable using the physical product. Act 1104 is performed, in some instances, by a system 100 utilizing processor(s) 102, storage 104, sensor(s) 110, AI model(s) 112, input/output system(s) 114, communication system(s) 116, and/or other components. In some implementations, the one or more suggested exercises are selected based on output of an artificial intelligence (AI) model configured to generate suggested exercise output based on usage context input or user information input. In some instances, the one or more suggested exercises performable using the exercise apparatus are presentable at the user device in a format that includes one or more of: textual, still graphics, video, audio, or audiovisual. In some implementations, the format is selected based on output of an AI model configured to generate content format output based on usage context input or user information input.

Act 1106A of flow diagram 1100 includes displaying a user prompt that prompts a user to provide user input at the user device indicating an intended use of the physical product or a desired result from using the physical product. Act 1106A is performed, in some instances, by a system 100 utilizing processor(s) 102, storage 104, sensor(s) 110, AI model(s) 112, input/output system(s) 114, communication system(s) 116, and/or other components. In some implementations, the intended use of the physical product or the desired result from using the physical product comprises one or more of general fitness or rehabilitation.

Act 1106B of flow diagram 1100 includes receiving the user input indicating the intended use of the physical product or the desired result from using the physical product. Act 1106B is performed, in some instances, by a system 100 utilizing processor(s) 102, storage 104, sensor(s) 110, AI model(s) 112, input/output system(s) 114, communication system(s) 116, and/or other components.

Act 1106C of flow diagram 1100 includes displaying additional content based on the intended use of the physical product or the desired result from using the physical product. Act 1106C is performed, in some instances, by a system 100 utilizing processor(s) 102, storage 104, sensor(s) 110, AI model(s) 112, input/output system(s) 114, communication system(s) 116, and/or other components.

Act 1108 of flow diagram 1100 includes displaying an interface for tracking usage of the physical product. Act 1108 is performed, in some instances, by a system 100 utilizing processor(s) 102, storage 104, sensor(s) 110, AI model(s) 112, input/output system(s) 114, communication system(s) 116, and/or other components. In some implementations, the interface comprises one or more user input fields for receiving one or more metrics related to usage of the physical product. Furthermore, in some implementations, the metrics comprise one or more of: duration of workout, calories burned, distance covered, repetitions performed, time spent in one or more positions, exercise activities/regimens completed, goals achieved, or biometric data exhibited during exercise. Still furthermore, the interface is configured to display usage statistics associated with a plurality of users who have used the physical product. The usage statistics selected for display may be selected based on usage context or user information associated with the user (e.g., via one or more AI models). In some instances, the interface is configured to display usage data associated with use of the physical product in a statistical analysis format.

In some instances, a method for linking a product to product information according to the embodiments of the disclosure may include one or more of the following steps: providing at least one identifier with a product, the identifier being scannable or otherwise comprising indicia allowing a user to enter information from the identifier into a website or mobile application; if the user has downloaded the mobile application, navigating the user upon scanning the identifier or entering identifier information to a corresponding content item in the website or mobile application; if the user has not downloaded the mobile application, navigating the user to a download site for the mobile application.

The foregoing description has mentioned at least some examples in which a system navigates to, surfaces, or otherwise presents particular content in response to scanning of a scannable identifier associated with a physical product. In some implementations, the system displays the particular content directly in response to the scanning of the scannable identifier. In some implementations, the particular content becomes queued for display or becomes displayable after one or more additional inputs or triggers is detected (e.g., after receiving particular user input, after detecting a particular sensor input or server signal, etc.).

A system and method for linking a product to product information according to embodiments of the disclosure addresses the problem of existing products being insufficiently instructive for a user, which leads users to at best have to search extensively for corresponding product information and training and at worst to improperly use the product. By providing a system and method as described, a user may be seamlessly connected to training information regarding a product by simply scanning an identifier on their mobile device.

Not necessarily all such objects or advantages may be achieved under any embodiment of the disclosure. Those skilled in the art will recognize that the disclosure may be embodied or carried out to achieve or optimize one advantage or group of advantages as taught without achieving other objects or advantages as taught or suggested.

The skilled artisan will recognize the interchangeability of various components from different embodiments described. Besides the variations described, other known equivalents for each feature can be mixed and matched by one of ordinary skill in this art to construct a system and method for linking a product to product information under principles of the present disclosure. Therefore, the embodiments described may be adapted to any type of product and any type of information for any suitable device or context.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

1. A system for linking a product to product information, comprising:
a physical product configured for use by a user; and
a scannable identifier associated with the physical product, the scannable identifier being accessible to the user, wherein the scannable identifier is configured to cause a user device to navigate to content related to use of the physical product in response to scanning of the scannable identifier with a scanning sensor associated with the user device.

2. The system of claim 1, wherein the scannable identifier is affixed to the physical product or integrated into the physical product.

3. The system of claim 1, wherein the scannable identifier comprises a quick response (QR) code.

4. The system of claim 1, wherein the physical product comprises an exercise apparatus.

5. The system of claim 4, wherein the content related to use of the physical product comprises one or more suggested exercises performable using the exercise apparatus.

6. The system of claim 5, the one or more suggested exercises being selected based on output of an artificial intelligence (AI) model configured to generate suggested exercise output based on usage context input or user information input.

7. The system of claim 5, wherein the one or more suggested exercises performable using the exercise apparatus are presentable at the user device in a format comprising one or more of: textual, still graphics, video, audio, or audiovisual.

8. The system of claim 7, the format being selected based on output of an AI model configured to generate content format output based on usage context input or user information input.

9. The system of claim 4, wherein the content related to use of the physical product comprises a user prompt that prompts the user to provide user input at the user device indicating an intended use of the physical product or a desired result from using the physical product.

10. The system of claim 9, wherein the intended use of the physical product or the desired result from using the physical product comprises one or more of general fitness or rehabilitation.

11. The system of claim 10, wherein the user input triggers display on the user device of additional content related to use of the physical product, the additional content being based on the intended use of the physical product or the desired result from using the physical product.

12. The system of claim 4, wherein the content related to use of the physical product comprises an interface for tracking usage of the physical product.

13. The system of claim 12, wherein the interface comprises one or more user input fields for receiving one or more metrics related to usage of the physical product.

14. The system of claim 13, wherein the metrics comprise one or more of: duration of workout, calories burned, distance covered, repetitions performed, time spent in one or more positions, exercise activities/regimens completed, goals achieved, or biometric data exhibited during exercise.

15. The system of claim 12, wherein the interface is configured to display usage statistics associated with a plurality of users who have used the physical product.

16. The system of claim 15, the usage statistics selected for display being selected based on usage context or user information associated with the user.

17. The system of claim 12, wherein the interface is configured to display usage data associated with use of the physical product in a statistical analysis format.

18. A system for linking a product to product information, comprising:
a physical product configured for use by a user;
a scannable identifier associated with the physical product, the scannable identifier being accessible to the user; and
a user device associated with the user, comprising:
one or more processors; and
one or more hardware storage devices storing instructions that are executable by the one or more processors to configure the user device to:
scan the scannable identifier with a scanning sensor of the user device; and
in response to detecting the scannable identifier, navigate to content related to use of the physical product.

19. The system of claim 18, wherein:
the physical product comprises an exercise apparatus,
the content related to use of the physical product comprises a user prompt that prompts the user to provide user input at the user device indicating an intended use of the physical product or a desired result from using the physical product, and
the instructions are executable by the one or more processors to further configure the system to:
display the user prompt in response to detecting the scannable identifier;
receive the user input indicating the intended use of the physical product or the desired result from using the physical product; and
in response to the user input, display additional content based on the intended use of the physical product or the desired result from using the physical product.

20. The system of claim 18, wherein the instructions are executable by the one or more processors to further configure the system to activate one or more sensors associated with the user device for tracking usage of the physical product.
